(19) United States
(12) Patent Application Publication
(10) Pub. No.: US 2022/0319717 A1
(43) Pub. Date: Oct. 6, 2022

(54) SYSTEMS AND METHODS FOR AUTOMATED IDENTIFICATION OF TARGET POPULATIONS FOR SYSTEM INITIATION

(71) Applicant: P3 Health Partners, Henderson, NV (US)

(72) Inventors: James A. HENDERSON, JR., Henderson, NV (US); Unmesh SRIVASTAVA, Monrovia, CA (US)

(73) Assignee: P3 Health Partners, Henderson, NV (US)

(21) Appl. No.: 17/708,353

(22) Filed: Mar. 30, 2022

Related U.S. Application Data
(60) Provisional application No. 63/169,383, filed on Apr. 1, 2021.

(51) Int. Cl. G16H 50/80; G16H 50/70; G16H 10/60

(52) U.S. Cl. CPC G16H 50/80; G16H 50/70; G16H 10/60

(57) ABSTRACT

A method for outputting new target populations for system initiation using a first machine learning model and a second machine learning model, the method comprising: receiving, user data; storing the user data; identifying a target population based on the user data to select groups of individuals not currently serviced by a specific provider; identifying a population criteria for the target population; applying a population criteria to target population user data of the target population; determining that the population criteria for the target population is above a first threshold population criteria; receiving initiated population data for an initiated population; and determining that the system initiation meets an improvement threshold probability to improve the population criteria by a second threshold amount.

### CROSS-REFERENCE TO RELATED APPLICATIONS

This application claims priority to U.S. Provisional Application No. 63/169,383 filed Apr. 1, 2021, the entire disclosure of which is hereby incorporated herein by reference in its entirety.

### TECHNICAL FIELD

Various embodiments of the present disclosure relate generally to identifying new populations for receiving population care management and more particularly to, systems and methods for automated identification of target populations for system initiation.

### BACKGROUND

Population care management is commonly understood as the process of improving clinical health outcomes for a defined group of individuals. For example, a population may be a specific age group (e.g., 55 years or older) residing in a specific region (e.g., Pima County, Ariz.). Because different populations may have different health outcomes or different healthcare challenges, there exists a need to identify populations and determine common effective treatments and strategies for improving the population health. In particular, there is a need to provide tools and resources to patients in order to prevent, manage, and navigate illness. There further exists a need to assist providers by removing barriers or time-costs on providers, for example, by reducing or limiting administrative processes and “paper-pushing,” so that providers are able to spend more time focusing on patients.

With improvements in cloud computing, data storage technology, and data collecting applications, more data is available for processing now than ever before. In the context of healthcare, analysis of large volumes of patient data in particular is critical to assisting healthcare providers with making well-informed decisions and ultimately improving the quality of healthcare provided to patients. It is well-known that data analytics in healthcare is especially challenging in the United States, due to not only the large increase in the volumes of data being collected and stored, but due to lack of standardization of data formats, reporting, and applications that typically may vary between healthcare practices, hospitals, cities and states. This type of data analytics is especially important for identifying regions or communities that may not be receiving adequate or acceptable standards of care. For example, health outcomes, numbers of wellness visits, emergency room visits, and other metrics may be monitored for particular populations and compared to other populations. In this way, different populations may be compared, and populations who have lower health outcomes and quality of health service may be identified as needing additional assistance.

Conventional techniques, including the foregoing, fail to provide an improved and effective data fabric structure for providing data analytics to providers and patients and for identifying populations that can benefit from system initiation.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for outputting new target populations for system initiation using a first machine learning model and a second machine learning model. In one aspect, an exemplary embodiment of a method may include: receiving, by one or more processors, user data associated with users from an external server via a secure network connection; storing, by the one or more processors, the user data on a cloud-based data storage system; identifying, by one or more processors, a target population based on the user data to select groups of individuals not currently serviced by a specific provider, wherein the target population is output by the first machine learning model that receives the user data, applies the user data to one or more of first model weights, first model biases, or first model layers, and outputs the target population; identifying, by one or more processors, a population criteria for the target population; applying a population criteria to target population user data of the target population; determining, by one or more processors, that the population criteria for the target population is above a first threshold population criteria; receiving initiated population data for an initiated population; and determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount. The target population may be based on a user data associated with a geographical area. The target population may be further based on user data demographics and Risk Adjustment Scores. The population criteria may output by a third machine learning model configured to receive the user data from plurality of databases and output the population criteria based on the user data. The population criteria may be a ratio of health events per predetermined number of individuals for the target population. The improvement probability may be based on comparing the target population criteria with a comparison population criteria improvement. The comparison population may be a simulated population. The simulated population may include historical population data from one or more previously initiated target populations. The second threshold amount may be a criteria minimum improvement to the population criteria to reach the first threshold population criteria.

In one aspect, an exemplary embodiment of a method for outputting new target population for system initiation using a first machine learning model and a second machine learning model, the method including: receiving, by one or more processors, user data associated with users from an external server via a secure network connection; storing, by the one or more processors, the user data on a cloud-based data storage system; identifying, by one or more processors, a population criteria for target populations; identifying, by one or more processors, a target population based on the user data and criteria to find groups of individuals not currently serviced by a specific provider, wherein the target population is output by the machine learning model that receives the user data, applies the user data to one or more of first model weights, first model biases, or first model layers, and outputs the target population; determining, by one or more processors, that the population criteria for the target population is above a first threshold population criteria; and determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount. The improvement probability may be based on comparing the target population criteria with a comparison population criteria improvement. The population criteria may be output by a third machine learning model configured to receive the user data from plurality of databases and output the population criteria based on the user data. The second threshold amount may be a criteria minimum improvement to the population criteria to reach the first threshold population criteria. The population criteria may be a ratio of health events per predetermined number of individuals for the target population.

An exemplary embodiment of a system for outputting new target populations for system initiation using a first machine learning model and a second machine learning model, the system including; at least one memory storing instructions; and at least one processor executing the instructions to perform a process including: receiving, by one or more processors, user data associated with users from an external server via a secure network connection; storing, by the one or more processors, the user data on a cloud-based data storage system; identifying, by one or more processors, a target population based on the user data to find groups of individuals not currently serviced by a specific provider, wherein the target population is output by the machine learning model that receives the user data, applies the user data to one or more of first model weights, first model biases, or first model layers, and outputs the target population; identifying, by one or more processors, a population criteria for the target population; applying a population criteria to the target population user data of the target population; determining, by one or more processors, that the population criteria for the target population is above a first threshold population criteria; receiving initiated population data for an initiated population; and determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount. The target population may be based on a user data associated with a geographical area. The target population may be further based on user data demographics and Risk Adjustment Scores. The population criteria may be outputted by a third machine learning model configured to receive, the user data from plurality of databases and output the population criteria based on the user data. The population criteria may be a ratio of health events per predetermined number of individuals for the target population. The improvement probability may be based on comparing the target population criteria with a comparison population criteria improvement.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for data transmission, according to one or more embodiments.

FIG. 2 depicts an exemplary flow diagram to identify target populations for system initiation, according to one or more embodiments.

FIG. 3 depicts a flowchart of an exemplary method of identifying target populations for system initiation, according to one or more embodiments.

FIG. 4 depicts an exemplary flow diagram to identify target populations for system initiation, according to one or more embodiments.

FIG. 5 depicts a flowchart of an exemplary method of identifying target populations for system initiation, according to one or more embodiments.

FIG. 6 depicts a flow diagram for training a machine learning model, according to one or more embodiments.

FIG. 7 depicts an example of a computing device, according to one or more embodiments.

### DETAILED DESCRIPTION OF EMBODIMENTS

According to certain aspects of the disclosure, methods and systems are disclosed for providing an automated process for identifying target populations for system initiation, wherein system initiation may include providing population health/care management services/healthcare related services to a target population. Traditionally, entering a new market to provide population health management services may have involved performing analysis of population data manually. There is a need to automate the process of locating new segments of the population to provide health management services. Accordingly, improvements in automating the process of finding population groups to provide health care services to are needed.

As will be discussed in more detail below, in various embodiments, systems and methods are described for using machine learning to analyze relevant data related to potential patients and to determine grouping of individuals for system initiation. The potential grouping of individuals may be referred to as target population groupings or target groups. System initiation, as used herein, may mean to provide updated population health management/care management/health care services to an identified target population. This may include, but is not limited to providing tools and resources to patients in order to prevent, manage, and navigate illness, and assisting providers by removing barriers or time-costs on providers. System initiation may include automated enrollment of the target population based on user data collected, as disclosed herein. By training one or more identification machine-learning models, e.g., via supervised, semi-supervised learning, or unsupervised learning to learn associations between potential patient data and patient data of populations groups who have been previously initiated, the trained identification machine-learning model may be used to identify populations groups for initiation in response to the input of potential patient data.

Further, in various embodiments, systems and methods are described for using machine learning to apply a criteria to the potential target populations. By training one or more criteria machine-learning models, e.g., via supervised, semi-supervised learning, or unsupervised, to learn associations between a plurality of databases and correlating the information in the databases with each other, the trained criteria machine-learning model may be used to correlate patient's data and determine a criteria (e.g., health related criteria, health criteria, or population criteria) for the patients and the overall target populations.

Further, in various embodiments, systems and methods are described for using machine learning to determine whether system initiation would improve a target population's criteria by a threshold value. By training one or more determination machine-learning models, e.g., via supervised, semi-supervised, or unsupervised learning, to learn associations between the target population's data and a plurality of other population's data that may have undergone system initiation, the trained determination machine-learning model may be used to find a previously initiated population that has similar geographic/demographic characteristics to the target population, determine a criteria (e.g., health related criteria) for both populations, and determine a probability (e.g. improvement threshold probability) that system initiation of the target population would improve the criteria by a certain threshold value.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “health care provider,” “health service provider,” “hospital,” “doctor's office” or the like generally encompass an entity or person involved in providing medical and health care services. As used herein, terms like “user” or “patient” generally encompasses any person or entity that may require or request a medical checkup, a medical examination, medical guidance, any type of medical assistance, or engage in any other type of interaction with a hospital, health provider, nurse, physician's assistant, or doctor. The term “browser extension” may be used interchangeably with other terms like “program,” “electronic application,” or the like, and generally encompasses software that is configured to interact with, modify, override, supplement, or operate in conjunction with other software. As used herein, terms such as “user data” or the like generally encompass patient data, or data pertaining to one or more medical patients. A “staging table” generally refers to a permanent database, data structure, or the like used to store temporary data for future processing. “Atomic data” generally refers to data in a data store, database or data warehouse that is at its lowest level of detail, e.g., data that cannot be broken down into smaller parts (e.g., a zip code may be considered “Atomic Data” because it cannot be broken down any further into another data element)

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use, a system described herein may be used to identify target populations for system initiation. The system may first receive external potential patient data from an external database associated with one or more health institutions, insurance companies, and/or healthcare providers (e.g., Cigna, Aetna, Anthem, Blue Cross Blue Shield, and so forth) and stored on a cloud-based data lake associated with a data fabric system, for example, Microsoft Azure Data Lake. The system may further organize the external potential patient data in a variety of ways. Next, the system may input the potential patient data from the data fabric system or an external database into a “population identifier model” to determine initial target population groups for potential system initiation. The inputted data may include information on potential patients such as geographical location, which health provider and insurance company is utilized by each individual, demographics of the potential patients, or the like. The target population identifier may be able to identify and organize groups of potential patients categorized by grouping such as geographical area and demographics. These groupings may be of individuals that are not currently under a specific companies' health plan and have certain health insurance plans.

According to implementations, an identification machine learning model may be used to categorize individuals into target populations. The system may categorize individuals by training a determination machine learning model to learn associations between potential patient data and patient data of populations groups who have been previously initiated. The system may next output these as “population data sets for target populations.” The system may then perform further analysis on these datasets to determine whether to initiate the target populations.

Next, the system may input one or more target population data sets into a criteria machine learning model. The criteria machine learning model may provide two outputs. First, the criteria machine learning model may be identify a population criteria. The population criteria may be a ratio, number, or percentage to describe a health statistic of the target populations. For example, the criteria may be a ratio of admitted patients comparted to non-admitted patients for a population. Alternatively the criteria could be number of certain health events occurring or repeating, such as how many diabetes patients are in the target population. The criteria machine learning model may output one or more criteria to utilize while analyzing the target populations. Second, the criteria machine learning model may apply the criteria to the potential target populations to identify an occurrence or value for the criteria in the potential target populations. This may be performed through machine learning. By training one or more criteria machine-learning models, to learn associations between a plurality of databases and correlating the information in the databases with each other, the criteria machine-learning model may output relevant patient medical data with the corresponding individuals within the target group. The criteria machine-learning model may output an applicable criteria for the target group, by applying the criteria to each individual within the target population. The output criteria may be a criteria that is above a threshold value (e.g., higher than threshold hospital admittance rates). Although an output criteria is generally discussed as above a threshold value, it will be understood that the output criteria may be above, below, or within a threshold range (e.g., number of patients that are not admitted to a hospital may be below a threshold to meet the criteria).

Next, the target groups, which have corresponding criteria's determined may be inserted into a determination machine learning model. The determination machine learning model may first may compare the criteria to a first threshold value to determine whether the criteria needs to improve for the target population (e.g., if the criteria for the target population is below a threshold amount). If the criteria is considered below (or in some cases above) the first threshold value for the target population, the system may determine that the system initiation for the target population is not required. The target population's criteria above (or in some cases below) the first threshold value may indicate that the potential target population has a criteria that requires improvement. Based on the criteria being above the first threshold, the potential target population may be identified as a target population for system initiation.

Next, the system may, utilize a determination machine learning model to determine whether initiating the target group may provide an improvement to the criteria of the target population (e.g., lower hospital admittance rates). This may be performed by inserting the target populations into a determination machine learning model. The determination machine learning model may utilize machine learning to determine whether system initiation may improve the criteria of the target population by a second threshold value. When the determination machine learning model meets a second threshold value/amount, the determination model may output that an improvement threshold probability may be met or above 50%. An improvement threshold probability may be defined as a probability that initiation may improve a criteria by second threshold value. The second threshold value may correspond to a minimum improvement to the criteria for a given target population to the first threshold value or to a value greater than or less than the first threshold value. This may be done by training the determination machine learning model that may be trained to find corresponding populations that have been previously initiated. The determination machine learning model may compare the effect of initiation on the corresponding populations to determine whether initiation improved the criteria by the second threshold value. The determination machine learning model may then output whether the target population's criteria may improve to the second threshold value, based on how the similar corresponding population groups historically responded to system initiation. In another embodiment, the corresponding population may be a simulated population that may be based on combining the populations of one or more past initiated groups of individuals. If the determination machine learning model determines that the target population's criteria may be improved by the second threshold value, the determination machine learning model may output initiation system initiation recommendation.

According to an example, the system may receive extensive patient data from the hospital and medical systems within the state of West Virginia. Further general patient data such as geographic, demographics, and health insurer information may be inputted into the system. The system may input this data into an internal database and organize the information within a data fabric system, as described herein. The organized data may then be inputted in an identification machine learning model within the system. Through machine learning, the identification machine learning model may, by applying the target population data to one or more of biases, weights, and/or layers, identify a section of the population similar to a previously initiated population group. The model may output a target population that is located in Morgantown, W. Va. and consist of 30,000 individuals. This may be based on the population having a similar geographical area and demographics of a previously initiated group. The system may then send the target population group in Morgantown to the criteria machine learning model. The criteria machine learning model may first identify a criteria such as the percentage of the population that has gone to the hospital or doctor's office for hypertension in a given year by applying the target population data to one or more of biases, weights, and/or layers. Next, the criteria machine learning model may correlate all medical records of individuals within Morgantown. This may, for example, include identifying hypertension cases in Morgantown from the different health providers in Morgantown. Next, the criteria machine learning model may determine that there are 4,000 patients who visited a doctor for cases of hypertension in Morgantown in the year of 2021. The criteria machine learning model may determine that the criteria score of the target population in Morgantown is 13.3%, for hypertension.

Next, the target population, with the criteria score may then be inputted into a determination model. The determination machine learning model may then compare the Morgantown criteria with a first threshold percentage of hypertension cases per year. For instance, the determination machine learning model may use a first threshold value of 7% for the criteria of percentage of the population hypotension cases per year. The first threshold value may be determined by the determination machine learning model based on, for example, hypertension cases nationwide and/or based on hypertension cases for populations similar to the population of Morgantown. The determination machine learning model may then determine that because the Morgantown population group has a hypertension value higher than the threshold value, that the Morgantown population is a candidate for system initiation. The determination model may then, by applying the target population data to one or more of biases, weights, and/or layers, compare the traits of the Morgantown's population data to all past initiated populations (e.g., the determination model may be trained to include information on all past initiated populations). The determination machine learning model may find that the Morgantown's population matches a previously initiated population from Fairmont, W. Va. The Fairmont, W. Va. may be previously initiated and the criteria data after initiation may be available to the determination machine learning model. The determination machine learning model may then analyze the effect of initiation on the Fairmont population group. The model may determine that prior to initiation, the Fairmont population group had a criteria of 14.7% and that after initiation, the criteria of Fairmont dropped to 6% on average in following years. In this example, the determination machine learning model may determine whether an improvement by a second threshold value is probable, to suggest system initiation. The second threshold value may correspond to a 5% improvement, meaning a decrease in the hypertension criteria by 5%, in order to suggest initiation. The determination model may determine that because the similar population of Fairmont improved its criteria by over 5% (the second threshold value) that the population group of Morgantown may be likely to also improve by a similar amount and thus the determination machine learning model may output Morgantown as a population for system initiation.

It should also be understood that the examples above are illustrative only. The techniques and technologies of this disclosure may be adapted to any suitable activity.

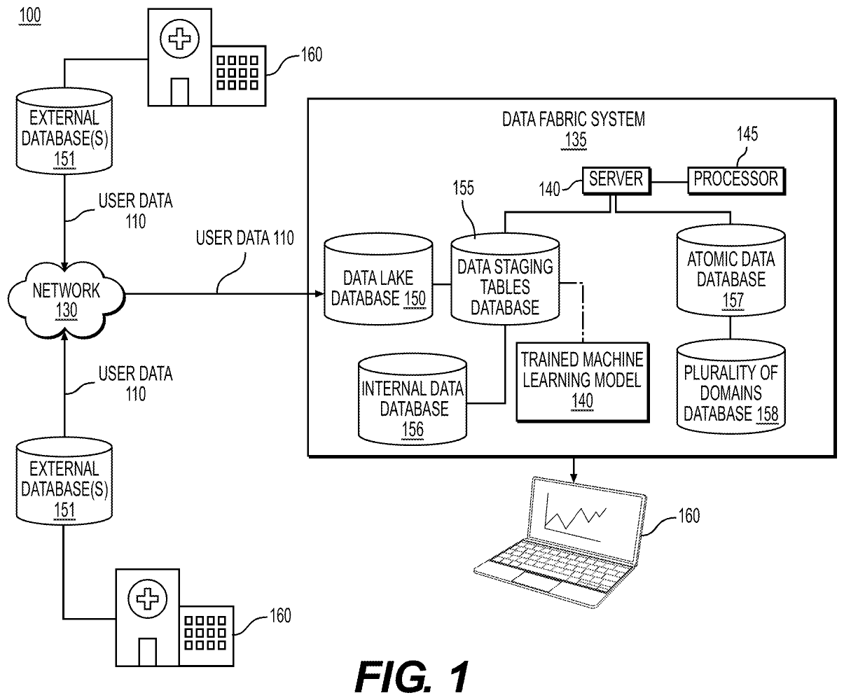

FIG. 1 depicts an exemplary environment, such as environment 100, which may be utilized with techniques presented herein. One or more healthcare institutions 170, external database(s) 151, and graphical user interface 160 (“GUI”) may communicate across an electronic network 130. One or more data fabric systems, for example, data fabric system 135, may communicate with one or more of the other components of the environment 100 across electronic network 130. The data fabric system 135 may according to some aspects of this disclosure comprise a processor 145, a server 144, a data staging tables database 155, a data lake database 150, an internal data database 156, a trained machine learning model 140, an atomic data database 157, and a plurality of domains database 158. The data lake database 150 may be a cloud-based database, and according to some aspects, may be located separately from the data fabric system 135. The graphical user interface 160 may be associated with a health care provider or a user, e.g., a user associated with one or more of generating, training, or tuning a machine-learning model for implementing a data fabric system, generating, obtaining and/or analyzing user data (e.g., patient healthcare data).

In some embodiments, the components of the environment 100 are associated with a common entity, e.g., a healthcare institution, a healthcare insurer, a population health management company, or the like. In some embodiments, one or more of the components of the environment is associated with a different entity than another. The systems and devices of the environment 100 may communicate in any arrangement. Systems and/or devices of the environment 100 may communicate in order to one or more of generate, train, or use a machine-learning model to implement a data fabric system, among other activities.

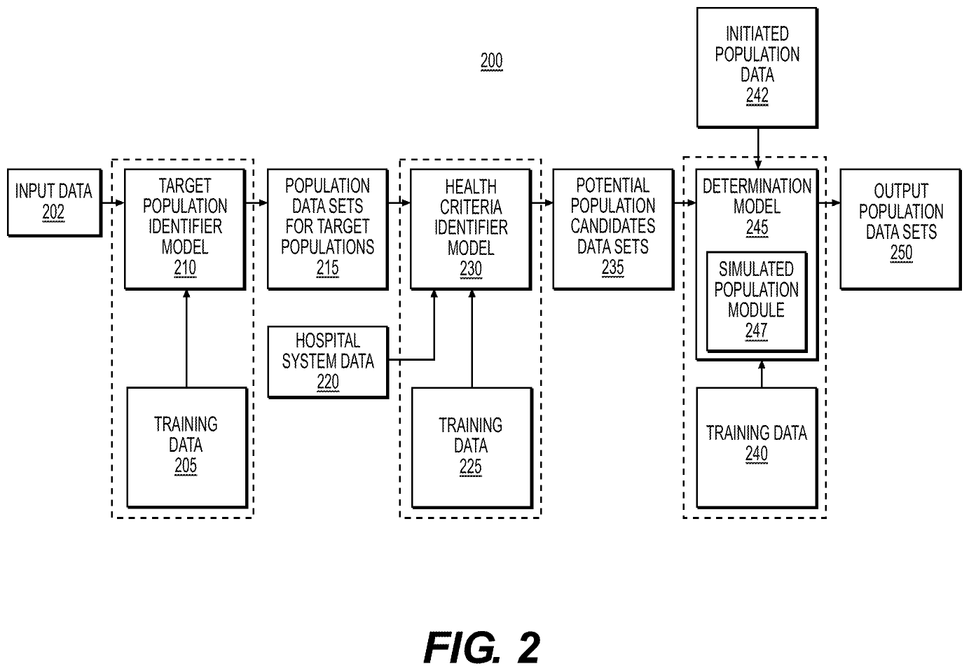

FIG. 2 depicts an exemplary flow diagram 200 for a system meant to identify target populations for system initiation, according to one or more embodiments. The system described in flow diagram 200 may provide input initial data 202 to a target population identifier model 210. Input data 202 may be filtered, modified, and/or otherwise manipulated and provided to a health criteria identifier model 230 and a determination model 245 to output population data sets 250 which may define target populations for system initiation.

Input data 202 may be organized and stored within data fabric system 135, as shown in FIG. 1. Input data 202 may, alternatively, be received from external data systems that include healthcare and general population information. Input data 202 may include, but is not limited to, data of individual's demographics, health data, geographical location, health care provider, insurance company and further identifying information for a group of individuals. The health data may include, but is not limited to, medical records such as diagnoses, lab test results, x-ray results, emergency room visit and discharge records, hospital or clinic admission information, and other information relevant to the health, medical treatment, and well-being of a user or patient. Health data may also include a Medicare risk adjustment factor “RAF” for individuals. The RAF may be a risk factor that is assigned to an individual by the Centers for Medicare and Medicaid Services (CMS), which may be utilized to describe the potential medical costs for an individual based on their demographic information and reported diagnoses. The RAF may take into account an individual's medical history, past surgical history, medical exam (that take temperature, heart rate, respiratory rate, and BP), average pain felt, and any other medical conditions.

Input data 202, may be input into a target population identifier model 210 (e.g., including an identification machine learning model, as further discussed herein). Target population identifier model 210 may be configured to identify groups of individuals (e.g., target groups) to perform system initiation. These target groups may be outputted as population data sets as target populations 215. The target groups may not currently be serviced by a particular health service provider and may be benefited by receiving new health care services via system initiation. Population data sets for target population 215, may include a subset of information (user data) included in the input data 202 for the selected individuals within the target group. Target population identifier model 210 may be configured to organize and output population data sets for target populations 215 in a variety of ways.

In one embodiment, target population identifier model 210 may create groups based on individual geographic location. For instance, the identified target populations may all be within a certain geographic area such as a certain town or county or a subset of the same. Alternatively the target group's geographic area may be organized by being located within a certain physical area, such as within a fifty mile radius. In another embodiment, the target groups may be further refined based on demographics. The target groups may also be based on the target group having certain RAF scores, such as having an average RAF score under a certain value. For instance, target population identifier model 210 may refine target groups by a geographic location first and then make sure that the population has a certain percentage of the population above a specific RAF score. Target population identifier model 210 may further have a requirement that a certain percentage of the population utilize particular insurance providers. Target population identifier model 210 may further refine target groups to be individuals with certain insurance plans or providers. Further, the target groups may require that a certain percentage such as 70% of the population have insurance from companies that would allow for initiation.

According to an implementation of the disclosed subject matter, a target population identifier model 210 may be implemented using an identification machine learning model. The identification machine learning model may be trained using supervised, semi-supervised, or unsupervised learning. The training may be based on training data 205 that includes a plurality of individuals (e.g., using user IDs), information associated with the plurality of individuals (e.g. health data, demographic data, location data, preference data, etc.), and/or historical target population information. The training may be conducted such that components of the target population identifier model (e.g., weights, biases, layers, etc.) are adjusted to output target populations based on the training data and/or one or more other criteria. The one or more criteria may include, for example, bounds on population size, diversity, geographical bounds, and/or contractual bounds. Identification machine learning model of target population identifier model 210 may be trained to output a target population based on the plurality of individuals, individual information, historical target population information, and/or other criteria.

Trained identification machine learning model of target population identifier model 210 may receive input data 202 including data associated with a number of potential individuals. The number of potential individuals may be from a plurality of different locations, from a contiguous location, or the like. Identification machine learning model of target population identifier model 210 may also receive inputs including one or more other criteria, as disclosed herein. Target population identifier model 210 may apply the received inputs and output one or more population data sets for target populations 215. Population data sets for target populations 215 may include subsets of the plurality of potential individuals. According to an implementation, the output of target population identifier model 210 may be one or more groups of individuals in one or more locations. Each of the one or more groups of individuals may be respective target populations that may be further analyzed, as disclosed herein, to determine whether to perform system initiation.

Next, population data sets for target populations 215 may be fed to a health criteria identifier model 230, having a criteria machine learning model. The criteria machine learning model of health criteria identifier model 230 may be capable of identifying a population criteria for the target populations 215. The criteria machine learning model of health criteria identifier model 230 may output potential population candidate data sets 235 that are identified based one or more criteria to the population data sets for target populations 215. As discussed earlier, criteria machine learning model of health criteria identifier model 230 may, first, determine a criteria and, second, apply the criteria to the target population to determine the target population's performance based on the criteria.

Criteria machine learning model of health criteria identifier model 230 may first determine one or more criteria (e.g., health criteria). The population criteria may be, for example, a ratio, a number, or a percentage. The population criteria may be based on, for example, a number of disease or condition occurrences, admittance rates, re-admittance rates, and/or health trends. For example, admitted patients (e.g., patients that are admitted to a hospital in a given amount of time) may be compared to patients that are not admitted (e.g., to determine a ratio), instances of certain health events among the population, rates of repetition for certain health events, or rates of repeated admittances for the individuals of a target groups. Example health events that may be utilized to determine a criteria may include, but are not limited to, cases of diabetes, hypertension, cancer, fatty liver disease, obesity, chronic kidney disease (CKD), depressive disorders (e.g. depression, persistent depressive disorder, bipolar disorder, seasonal affective disorder, psychotic depression, peripartum depression, premenstrual dysphoric disorder), asthma, etc. The criteria may also be based on the amount of specialists or hospital visits by individuals within the target groups. In another embodiment, the criteria may consider cost and/or quality of care, in addition to or as an alternative to health events. Quality of care may refer to the degree to which health services for individuals and populations increase the likelihood of desired health outcomes. An example criteria ratio could be the amount of hospital visits that take place in a year divided by the total population of the population data sets for target population 215. Health criteria identifier model 230 may select the criteria in a variety of ways. In one embodiment, health criteria identifier model 230 may be implemented by utilizing a specific predetermined criteria. In another embodiment, the flow diagram 200 may allow for a user to select a criteria from the variety of options. In another embodiment, the criteria may be determined by the criteria machine learning model of health criteria identifier model 210 based on the initial input data 202. Criteria machine learning model of health criteria identifier model 230 may be trained using supervised, semi-supervised, or unsupervised learning. The training may be based on training data 225 and/or hospital system data 220 that includes a number of health events (e.g. cases of hypertension, diabetes, etc.), hospital admission, readmission rates of the population data sets for target population 215 and/or the like. The training may be conducted such that components of the target population identifier model 210 (e.g., weights, biases, layers, etc.) are adjusted to output criteria based on the training data. The criteria machine learning model of health criteria identifier model 230 may be trained to select a criteria based on the available information related to the criteria such as the plurality of individuals within the target population, the individual's available information within the target population, past criteria's utilized, and/or other criteria. The trained criteria machine learning model of health criteria identifier model 230 may receive inputs including data related to target population data sets for target population 215. The target population data sets may include information on all relevant health events, hospital admittances, hospital re-admittance, medical deaths, etc. Trained health criteria identifier model 230 may apply the received input and output a criteria to utilize. According to an implementation, the criteria may be based on the system selecting the criteria which the system has the most information on or a criteria most relevant to a given target population (e.g., based on an occurrence or lack of occurrence of instances associated with the criteria).

Next, criteria machine learning model of health criteria identifier model 230 may then analyze the selected criteria of population data sets for target population 215 to determine an output criteria. This may be determined based on accessing a plurality of databases and correlating the information in the databases with each other. These databases may include hospital system data 220 and initial input data 202 related to the individuals. For example, a given patient may be admitted at a first hospital and a second urgent care within the span of two months. Accordingly, health criteria identifier model 230 may receive data from a first hospital system and a second urgent care system (e.g. hospital system data 220). Health criteria identifier model 230 may determine that separate data from the first hospital system and second urgent system correspond to the same patient. The correlation may be made, for example, by an identification machine learning model, as disclosed herein. The criteria machine learning model may receive, as inputs, data from different health care systems and extract patient data (e.g. demographic data, identifying data, medical data, treatment data, etc.). Weights, biases and/or layers of the identification machine learning model within the health criteria identifier model 230 may be trained to identify correlation values for one or more patients, across the health care system data. The training may be conducted such that components of health criteria identifier model 230 (e.g., weights, biases, or layers) are adjusted to output target populations based on training data 225 and/or one or more other criteria. A correlation that meets a threshold correlation may result in a match output by the identification machine learning model. Accordingly, matched output patient data for a given patient may be associated with the given patient. Population criteria for the given patient may be based on the matched output patient data. Population criteria for a target population that includes the given patient may be generated by single patient data or matched patient data for that target population. The criteria machine learning model of health criteria identifier model 230 may output potential population candidate data sets 235, which includes population data sets for target population 215 and the criteria (output by the criteria machine learning model of heath criteria identifier model 230) for the target population.

In another embodiment, the system represented by flow diagram 200, may receive inputs of population data sets for target population 215 externally, which can then be inputted into health criteria identifier model 230. In this embodiment, the system may be fed particular population grouping to be further analyzed to determine a criteria and whether initiation could benefit the criteria. For example, the system may be utilized once a potential population grouping has been determined externally of the system, to verify whether providing certain health services my increase a criteria of the population and to determine how large of an increase (or decrease, depending on the criteria) this may be.

Next, potential population candidate data sets 235 may be inputted into a determination machine learning model of determination model 245. The determination machine learning model of determination model 245 may be configured to, first, determine whether the potential population candidate data sets 235 have criteria values above a threshold first/initial criteria (alternatively, the criteria values may be below a certain threshold or within a threshold range, depending on the criteria selected). Second, the determination machine learning model of determination model 245 may determine whether system initiation may adjust the criteria by a second threshold value. The second threshold amount may be a criteria minimum improvement to the population criteria to reach the first threshold population criteria. Criteria minimum improvement may be the required expected improvement of a criteria for a target group based on initiation. This determination may indicate that a particular potential population candidate data set 235 is a candidate for a system initiation, as further discussed herein. The determination machine learning model of determination model 245 may output an indication or a probability that a system initiation would improve the population criteria for the target populations by a second threshold amount. The determination machine learning model of determination model 245 may output a “improvement threshold probability,” which is a percentage chance that the initiation will improve the criteria by the second threshold amount.

First, the determination machine learning model of determination model 245 may determine whether a criteria for a potential population candidate data set 235 is above or below a first/initial threshold value. Determination model 245 may use a threshold value that corresponds for each potential criteria output by the criteria machine learning model of health criteria identification model 230. The determination machine learning model may compare the criteria for each potential population candidate data set 235 with the initial threshold value. In one embodiment, if the criteria is above the first threshold value, then the potential population candidate data sets 235 may be sent for further analysis to determine whether system initiation may take place. If the criteria is below the second threshold value, then this target group may not be considered for system initiation. In another embodiment, if the criteria is below the first threshold value, then the potential population candidate data sets 235 may be sent for further analysis to determine whether system initiation may take place. If the criteria is above the second threshold value, then this target group may not be considered for system initiation.

For example, the re-admittance rate (e.g. an example criteria) of patients that are re-admitted to a hospital within two months from a first admittance may be determined for a target population by the criteria machine learning model of health criteria identifier model 230. This rate may be 20% for an example population. The determination machine learning model of determination model 245 may determine if the criteria (e.g., the re-admittance rate) exceeds a predetermined threshold value. In this example, the threshold value may be 12%. In this example, with the criteria being re-admittance rate, because the criteria (20%) for the target group is above the threshold value (12%), the determination model 245 may further analyze the potential population candidate data sets 235. If the criteria for a target group was lower than the threshold value (12%), for example if it was 10%, then the target group may no longer be considered for system initiation based on the re-admittance rate criteria.

If the criteria is above the threshold value, determination model 245 may be triggered to determine whether a system initiation is likely to reduce the re-admittance rate by a second threshold amount, such as dropping the rate at least 9%. The second threshold value may be a predetermined threshold value corresponding with each potential criteria or may be determined by the determination machine learning model. The second threshold value may represent a value, ratio, presentation, etc. of change of the criteria is necessary benefit from system initiation. The determination machine learning model of determination model 245 may perform this analysis by first identifying a comparable population to the target group (preferably one that has been previously initiated) and, second, analyzing whether system initiation improved the comparable population criteria by a second threshold value.

The determination machine learning model of determination model 245 may receive potential population candidate data sets 235, which may include population data such as demographic information, medical information, geographic information, and treatment information. The determination machine learning model of determination model 245 may correlate data from different sources, as discussed above, to generate the population data for the target groups. Population attributes such as demographic information, medical information, treatment information, and insurance information may be extracted from the potential population candidate data sets 235. The determination machine learning model may receive potential population candidate data sets 235 and initiated population data 242, and may be trained using training data 240. The initiated population data 242 may be population data related to a plurality of other populations that underwent system initiation. Initiated population data 242 may include all historical population data from one or more previously initiated target populations. Historical population data may include any type of data that input data 202 includes such as individual's demographics, health data, geographical location, health care provider, insurance company and further identifying information for a group of individuals. Initiated population data 242 may include population data from population groups that use certain health care providers or insurance. The data for populations that have undergone system initiation may include information on the changes in population criteria. As an alternative, the determination model's 245 machine learning model may be previously trained based on the plurality of other populations, instead of receiving related data as inputs.

According to an implementation of the disclosed subject matter, a determination model 245 may be trained using supervised, semi-supervised, or unsupervised learning. The training may be based on training data 240 that includes a plurality of individuals (e.g., using user IDs), information associated with the plurality of individuals (such as health data, demographic data, location data, preference data, etc.), historical target population information. Further, initiated population data 242 may be utilized for training. The training may be conducted such that the components of the determination machine learning model (such as weights, biases, or layers) are adjusted to output comparable populations that are determined to be similar (e.g., based on population data) to potential population candidate data sets 235. For example, the comparable populations may have similar bounds on population size, diversity, geographical bounds, and/or contractual bounds. Additionally, the comparable populations may have information related to potential criteria's that may be utilized by the system described in flow diagram 200.

Determination model 245 determination machine learning model may compare the population attributes for the potential population candidate data sets 235 with the attributes of one or more other populations. Determination model 245 determination machine learning model may identify comparable populations to compare to potential population candidate data set 235. The comparable populations may be populations that overlap or otherwise resemble the target population by a threshold amount. In one embodiment, the comparable populations may have similar geographical and demographic grouping. Importantly, any output comparable population should have information on the criteria of the comparable population and how the criteria has changed over time.

Once determination model's 245 determination machine learning model has identified one or more comparable population data set to compare with potential population candidate data sets 235, the population criteria (output by criteria machine learning model of criteria identifier model 230) may be identified for the comparable population. Next, the change to the population criteria, such as re-admittance rate, for the comparable population may be identified. The change to the criteria may be calculated by comparing the difference in the criteria prior to system initiation and after system initiation.

In another embodiment, the comparable population may be a simulated population that is created by a simulated population module 247 of determination model 245. The simulated population may overlap or otherwise resemble potential population candidate data sets 235 that are inputted to determination model 245. In one embodiment, simulated population module 247, may create a simulated population by combining individuals from multiple initiated population data 242 in order to create a simulated population that resembles the characteristics of the target population in potential population candidate data sets 235. Specifically, some of the similar characteristics may be the health data, demographic data, location data, and preference data. Accordingly, the population criteria (e.g., re-admittance rates) for the simulated population may be identified. The population criteria for the simulated population may be determined for the initial simulated population and for the simulated population after initiation was performed/assumed.

Determination model 245 may compare criteria for potential population candidate data sets 235 with the simulated or comparable population data criteria to determine whether system initiation should occur. According to the implementations discussed above, if a comparable or simulated population's criteria is improved by the second threshold amount, then a corresponding potential target population may be output as a target population. The target population's data may be output as output population data sets 250. In one embodiment, output population data set 250 may include a probability that a system initiation would improve the population criteria for the target population by the second threshold amount. As discussed earlier, the second threshold amount may be a ratio, percentage or number value for each applicable criteria. The second threshold value may be determined by determination machine learning model. In another embodiment, the second threshold value may be equal to the difference between the first threshold value for a given criteria and the actual value for the criteria for a potential population candidate data set. For example, if the criteria value for admittance rates is 20% for admittance rates to a hospital for a potential population and the first threshold value is 12%, then the second threshold value may be 9% (e.g., to reach below the first threshold value of 12%). This may mean that the probability that the criteria value of the target group improves by 9% is above a probability threshold, in order to output a system initiation recommendation.

The automated nature of identifying populations where criteria may be improved, may allow for a reduction in cost, time, and resources expended in identifying target population. Determination model 245 may not implement system initiation when determination model 245 determines, based on comparison to a comparable or simulated population, that the criteria for the potential population candidate data set 235 will not be improved by a second threshold. For example, if based on comparing to a similar population that was initiated, it is unlikely for system initiation to decrease a hospital re-admittance rate by a large enough value (a second threshold value), such as brining down a percentage for 20% to 11%, then system initiation will not be suggested and the output population data sets 250 will not include the analyzed potential population candidate data sets 235.

Output population data sets 450 may organize information related to the potential target group in a variety of ways. The output population data sets 450 may include a list of all of potential individuals within the target group. The list may be a file such as an .xls file; a csv file; a pipe delimited file, or a text file. The list may include all initial input data that corresponds with each individual. Further, output population data sets 450 may be refined to include/exclude certain input data. Additionally, the criteria for the target group may be included in the output population data set. The output population data sets 450 may be inputted into data fabric system 135 or another external database.

System initiation may include providing a transition of health care services to a target population. The new health care services may include, but are not limited to, providing increased educational resources, improving tracking of patient data, allowing for primary care providers to have access to medical records, and performing care management for patients with diseases such as diabetes. The new health care services may allow for overall lower healthcare costs for the target population. Initiation may include “automated enrollment” into care management. Automated enrollment may include notifying the target population and/or the insurance companies that represent the target population about the opportunity to provide updated health services.

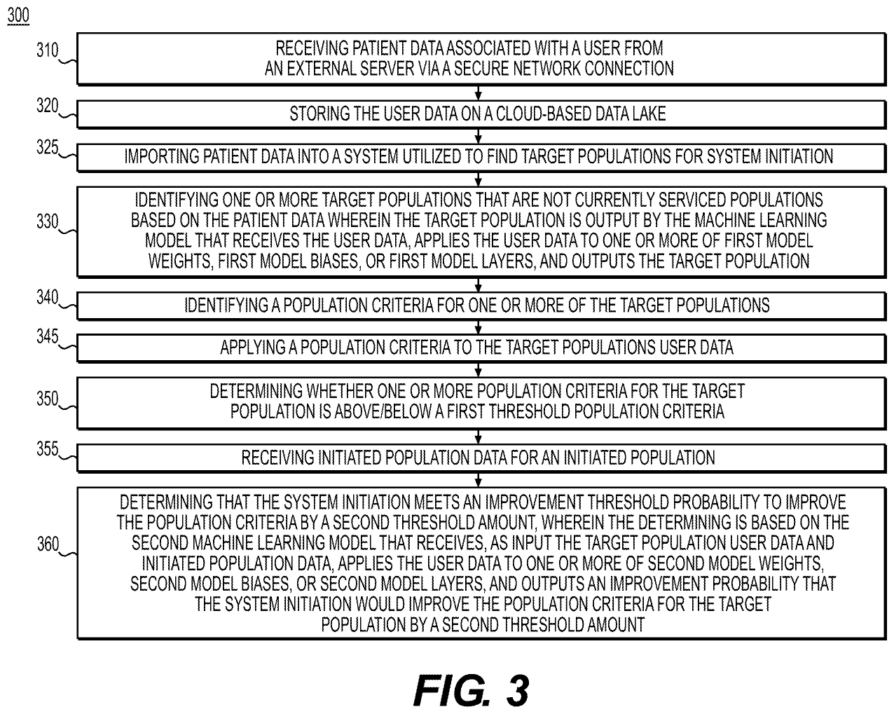

FIG. 3 illustrates a flowchart of an exemplary method of identifying target populations for system initiation, according to one or more embodiments, such as in the various examples discussed above in FIG. 2. At step 310 of the flowchart 300, data fabric system 135 may receive user data 110 from an external server via a secure network connection. According to some aspects, user data 110 comprises one or more of: user institution records, user identification information, or user financial data. User data 110 may be, for example, patient data, including data relevant to a patient or user's health medical records or history. For example, user data 110 may include medical records such as diagnoses, lab test results, x-ray results, emergency room visit and discharge records, hospital or clinic admission information, and other information relevant to the health, medical treatment, and well-being of a user or patient. According to some aspects, the user data 110 is in the format of one or more of: an .xls file; a csv file; a pipe delimited file, or a text file. At step 320, user data 110 may be stored and organized within data fabric system 135.

At step 325, target population identifier model 210 may receive user data 202 from data fabric system 135. In another embodiment, identifier model 210 may receive user data 202 from an external database.

At step 330, flow diagram 200 may identify a target population that is not currently a serviced population by scanning patient data associated with a geographical area and identifying gaps in the data corresponding to groups of patients. This may be done by target population identifier model 210 identifying population data sets for target populations 215. Target population identifier 210 may analyze input data to determine potential populations based on, but not limited to, geographic, demographics, health care providers, and insurance providers. Target population identifier model 210 may utilize machine learning techniques to develop the population data sets for target populations 215. The target populations, for which population data sets for target population 215 are created, may be groups that will be further considered for system initiation.

In step 340, the system represented in flow diagram 200 may identify a criteria for one or more of the population data sets for target population 215. The system represented in flow diagram 200, may identify a population criteria through a health criteria identifier model 230. Health criteria identifier model 230 may receive population data sets for target populations 215 from a target population identifier model 210. Alternatively, population data sets for target population 215 may be provided and inserted externally into health criteria identifier model 230 through an external software. As discussed herein, the criteria may be a ratio, number, or percentage based on instances of health events or repeat health events. Health criteria identifier model 230 may be programmed to use a certain pre-determined criteria. In another embodiment, the criteria may be determined utilizing machine learning techniques to determine a criteria that based on having the most data on the criteria for all individuals across the population data sets for target populations. In another embodiment, a user may input which criteria should be utilized by the health criteria identifier model 230.

At step 345, health criteria model 230 may apply the criteria to the target group. The health criteria model 230 may utilize machine learning techniques in order to correlate the data from different databases to correctly provide output patient data for the members of the target population. The machine learning system may receive as input, data from different health system data 220 and input data 202 and output matched patient data for each patient in the target group. Next, health criteria model 230 may determine the criteria of the target group and export this data as potential population candidate data sets 235.

At step 350, the system may determine whether one or more population criteria for a target population is below/above a first threshold value. The threshold values for criteria may be saved in determination model 245. Each criteria may have a corresponding first threshold value. For instance, a health criteria model 230 may select the criteria “hospital admittance percentage” which may be the percent of a target group population that was admitted to a hospital within a calendar year. Health criteria identifier model 230 may give a criteria score of 7% to a target group which is then exported as a potential population candidate data set 235. Determination model 245 may determine that the first threshold value for the criteria of “hospital admittance percentage” is 5%. Determination model 245 may note that because the criteria of 7% is greater than the first threshold value of 5%, the system may continue performing analysis to determine whether system initiation may be suggested. If the criteria of the target group was 4% in this example, then the system may not suggestion the target group for system initiation. In other embodiments, for certain threshold values, determination model 245 may require that the criteria be below the first threshold value instead of above the first threshold value.

At step 360, the system may determine whether system initiation would improve the population criteria for a target population by a second threshold amount. Determination model 245 may utilize machine learning to determine whether a system initiation is like to reduce the criteria by a second threshold value. The machine learning may receive, as inputs, population data for the target population and population data from a plurality of other population, where the plurality of other populations may have previously been initiated. Further, determination model 245 may receive the change of criteria for the plurality of other populations that have been previously initiated. The machine model learning may compare population attributes for the target population with population attributes from the plurality of other populations and output a comparable population.

In another embodiment, the comparable population may be a simulated population created by a simulated population model 247. The simulated population may be a simulated population that is meant to have similar patient data to potential population candidate data sets 235. The simulated model may be made up of groups of individuals from multiple past initiated populations. The system representing flow diagram 200 may have a preference to utilize a comparable population, however, if there are no corresponding comparable populations, the system may then create and utilize a simulated population.

Determination model 245 may determine whether to suggest system initiation based on comparison between the patient criteria for the target population and the changes to the population criteria for the comparable population. If the comparable population has a criteria change by a second threshold value, then the system may suggest system initiation.

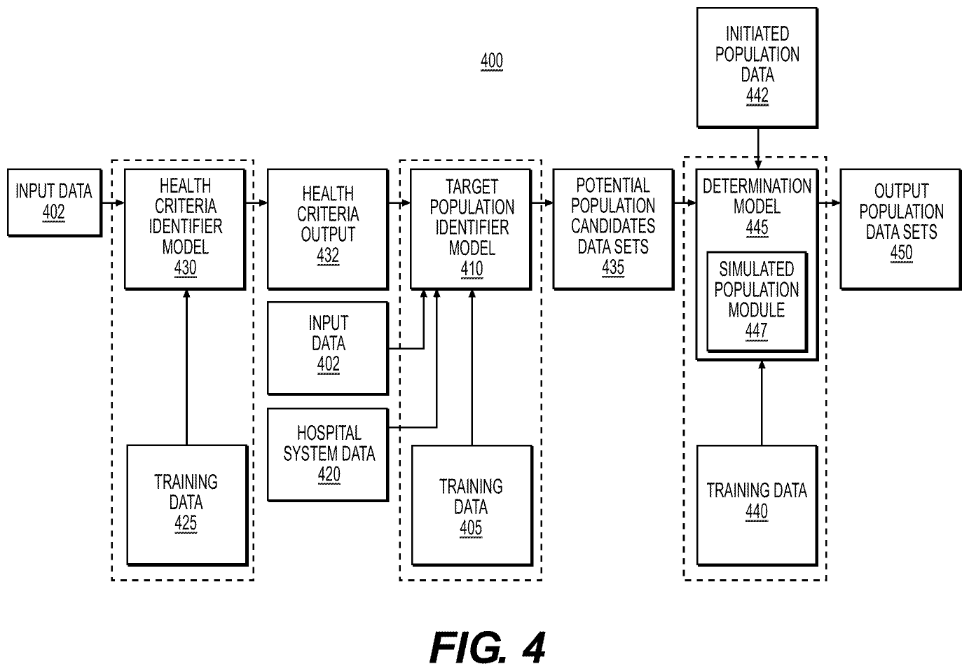

FIG. 4 depicts an exemplary flow diagram 400 to identify target populations for system initiation, according to one or more embodiments. The system described by flow diagram 400, may generally perform the same function as flow diagram 200 described in FIG. 2. Flow diagram 400 may include a health criteria identifier model 430, input data 402, a target population identifier model 410, and a determination model 445 that outputs population data sets 450 which may define target populations meant for system initiation. Flow diagram 400 may have the same input data 402 and potential output population data sets 450 as the input data 202 and output population data sets 250 from flow diagram 200.

Flow Diagram 400 may differ from flow diagram 200 such that flow diagram 400 may first determine a criteria prior to determining a target group. The criteria may then be utilized as a factor by target population identifier 410 when determining the initial target groups. As discussed earlier, health criteria identifier model 230 has two responsibilities, first determining a criteria and second applying the criteria to the target population. In contrast, the health criteria identifier model 430 only determines a criteria. The model does not apply the criteria to a target group. Health criteria identifier model 430 may be trained and the criteria may be determined in all the ways that health criteria identifier model 230 may be trained and utilized. Training data 425 may be equivalent to training data 225.

Additionally, for health criteria identifier model 430, rather than outputting a criteria as applied to a target group, like in flow diagram 200, may only output a health criteria output 432. Health criteria output 432 may be inputted into target population identifier model 430.

The second difference between flow diagrams 200 and 400 may be that target population identifier model 410 may utilize health criteria output 432 when determining target groups that will be outputted as potential population candidate data sets 435. Target population identifier model 410 may thus determine target groups in all of the same ways as target population identifier model 230, however, the target groups may require that the criteria be above or below a certain threshold value in order to be selected. Target population identifier model 410, may still be trained in all of the ways target population identifier model 210 may be trained. The training may be based on training data 405 and/or hospital system data 420 that includes a number of health events. Training data 405 may be equivalent to training data 205 and hospital system data 420 may be equivalent to hospitals system data 220. For example, target population identifier model 430 may refine a target group search to make sure that the “hospital re-admittance rate” is 7% or greater whenever selecting/searching for a target group. Target population identifier model 430 may store a first threshold value for each potential health criteria output 432. Target population identifier model 430 may also store whether the target group must have a criteria above or below the first threshold value in order to be output as a potential population candidate data set 435.

Target population identifier model 430 may also include an identification machine learning model similar to the identification machine learning model for health criteria identifier model 230 applying the health criteria output 432 to the potential target populations.

Determination model 445 may be similar to the determination model 245 of flow diagram 200. Similarly, initiated population data 442, simulated population model 447 training data 440 may be the same as the corresponding parts of flow diagram 200. Further, output population data sets 450 may provide the same output data/files as 250, however, the new system may lead to different outputs than flow diagram 200.

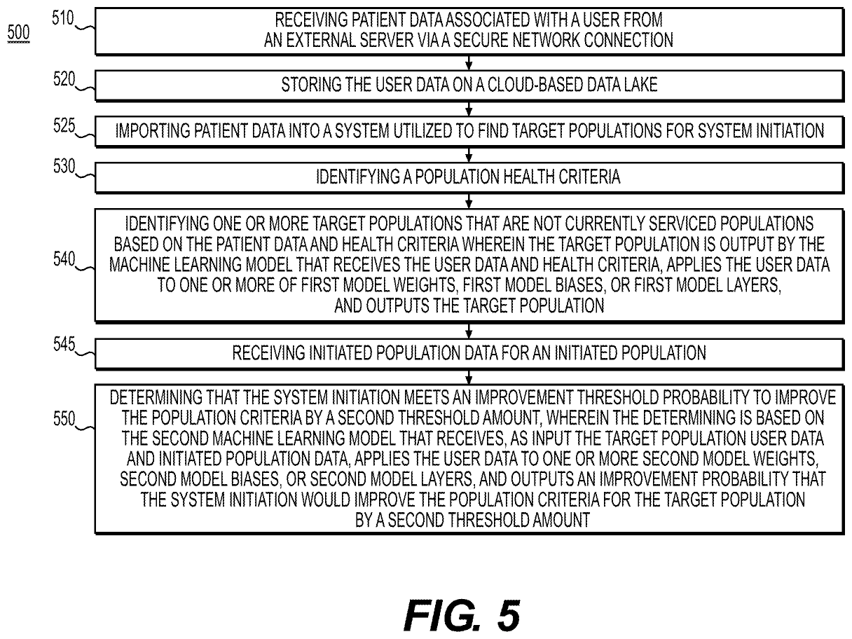

FIG. 5 depicts a flowchart of an exemplary method of locating target populations for system initiation, according to one or more embodiments, such as in the various examples discussed above in FIG. 4.

Steps 510, 520, and 525 may correspond to steps 310, 320, and 325 from FIG. 3. Step 530, identifying a population criteria may correspond to step 340 from FIG. 3. Step 530 may be differentiated from step 340 in that a population criteria is identified based on all provided data, not just the data of specific target populations. Health criteria identifier model 430 may be programmed to use a certain pre-determined criteria. In another embodiment, the criteria may be determined utilizing machine learning techniques to determine a criteria that is selected based on there being the most available information (based on input data 402) related to chosen criteria. In another embodiment, an external software may input which criteria should be utilized by health criteria identifier model 430. Health criteria output 432 may then be inputted into target population identifier model 430.

At step 540, target population identifier model 430 may determine target populations that are not currently serviced populations based on a population criteria and by patient data such as geographic, health, and demographic data. Target population identifier model 410 may create target potential population candidate data sets 435 in all of the same ways as target population identifier model 210 creates population data sets for target population 215, with the exception that target population identifier model 410 may filter potential target groups based on health criteria output 432. This may mean that if a potential target group does not average a predetermined criteria threshold value, that the target group may not be outputted as a potential population candidate data sets 435. This filtering by criteria may be similar to step 350 from FIG. 3, however, the target population identifier model 410, not the criteria identifier model 430 may perform the analysis.

At step 550, the system may determine whether a system initiation would improve the population criteria for a target population by a threshold amount (e.g., a second threshold amount discussed herein). Step 550 may be correspond step 360 from FIG. 3.

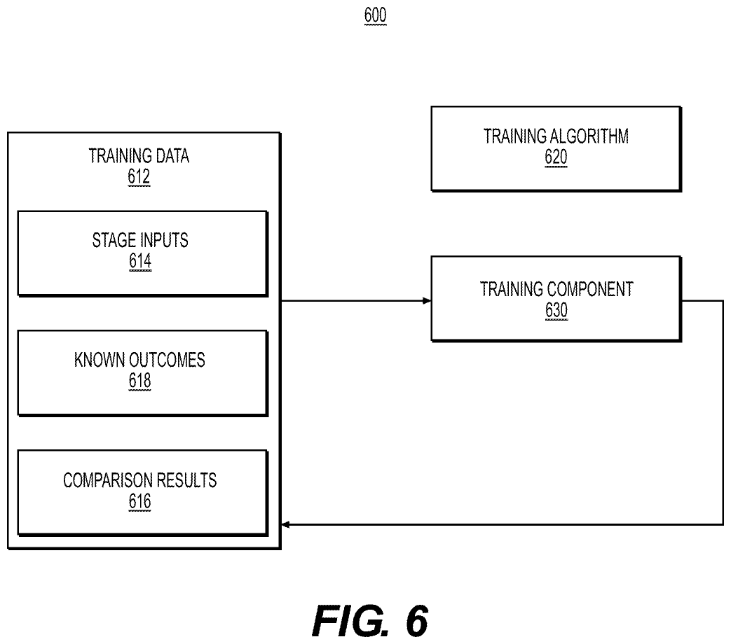

FIG. 6 depicts a flow diagram for training a machine learning model to implement a targeted medical outreach, according to one or more embodiments. One or more implementations disclosed herein may be applied by using a machine learning model. A machine learning model as disclosed herein may be trained using the flow diagram 200 of FIG. 2, flowchart 300 of FIG. 3, flow diagram 400 of FIG. 4, and/or flow chart 500 of FIG. 5. As shown in flow diagram 600 of FIG. 6, training data 612 may include one or more of stage inputs 614 and known outcomes 618 related to a machine learning model to be trained. The stage inputs 614 may be from any applicable source including a component or set shown in FIG. 1, 2, 3, 4, or 5. The known outcomes 618 may be included for machine learning models generated based on supervised or semi-supervised training. An unsupervised machine learning model might not be trained using known outcomes 618. Known outcomes 618 may include known or desired outputs for future inputs similar to or in the same category as stage inputs 614 that do not have corresponding known outputs.

The training data 612 and a training algorithm 620 may be provided to a training component 630 that may apply the training data 612 to the training algorithm 620 to generate a trained machine learning model 230 or any of the trained models within the target population identifier model 210, 410, health criteria identifier model 230, 430, or determination model 245, 445. According to an implementation, the training component 630 may be provided comparison results 616 that compare a previous output of the corresponding machine learning model to apply the previous result to re-train the machine learning model. The comparison results 616 may be used by the training component 630 to update the corresponding machine learning model. The training algorithm 620 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 600 may be a trained machine learning model such as the models used within the target population identifier model 210, 410, health criteria identifier model 230, 430, or determination model 245, 445.

It should be understood that embodiments in this disclosure are exemplary only, and that other embodiments may include various combinations of features from other embodiments, as well as additional or fewer features. For example, while some of the embodiments above pertain to implementing an automated outreach, any suitable activity may be used. In an exemplary embodiment, instead of or in addition to automated outreach to a patient, implementing a targeted medical outreach may include providing input to a medical provider's GUI.

In general, any process or operation discussed in this disclosure that is understood to be computer-implementable, such as the processes illustrated in FIGS. 1, 2, 3, 4 and 5, may be performed by one or more processors of a computer system, such any of the systems or devices in the environment 100 of FIG. 1, 200 of FIG. 2, or 400 of FIG. 4 as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1, FIG. 2, or FIG. 4. One or more processors of a computer system may be included in a single computing device or distributed among a plurality of computing devices. A memory of the computer system may include the respective memory of each computing device of the plurality of computing devices.

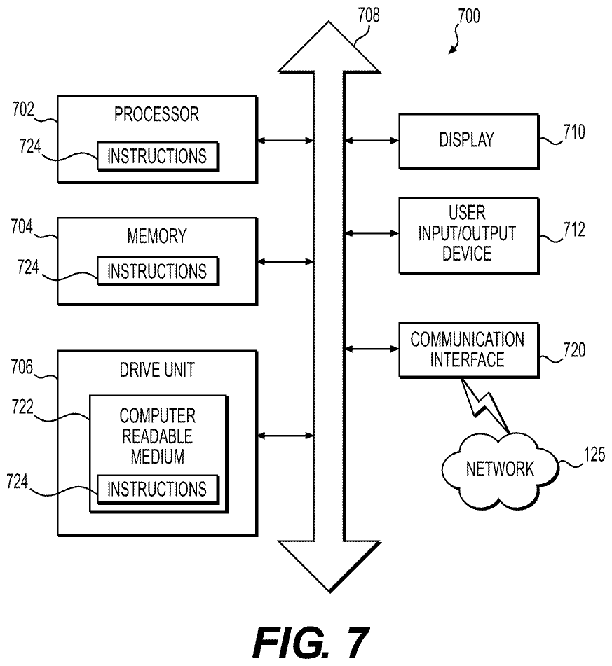

FIG. 7 is a simplified functional block diagram of a computer 700 that may be configured as a device for executing the method of FIGS. 3 and 5, according to exemplary embodiments of the present disclosure. For example, the computer 700 may be configured as the data fabric system 135, the target population identifier model 210, 410, the health criteria identifier model 230, 430, the determination model 245, 445, the simulated population model 247, 447, and/or another system according to exemplary embodiments of this disclosure. In various embodiments, any of the systems herein may be a computer 700 including, for example, a data communication interface 720 for packet data communication. The computer 700 also may include a central processing unit (“CPU”) 702, in the form of one or more processors, for executing program instructions. The computer 700 may include an internal communication bus 708, and a storage unit 706 (such as ROM, HDD, SDD, etc.) that may store data on a computer readable medium 722, although the computer 700 may receive programming and data via network communications. The computer 700 may also have a memory 704 (such as RAM) storing instructions 724 for executing techniques presented herein, although the instructions 724 may be stored temporarily or permanently within other modules of computer 700 (e.g., processor 702 and/or computer readable medium 722). The computer 700 also may include input and output ports 712 and/or a display 710 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A method for outputting new target populations for system initiation using a first machine learning model and a second machine learning model, the method comprising:
receiving, by one or more processors, user data associated with users from an external server via a secure network connection;
storing, by the one or more processors, the user data on a cloud-based data storage system;
identifying, by one or more processors, a target population based on the user data to select groups of individuals not currently serviced by a specific provider, wherein the target population is output by the first machine learning model that receives the user data, applies the user data to one or more of first model weights, first model biases, or first model layers, and outputs the target population;
identifying, by one or more processors, a population criteria for the target population;
applying a population criteria to target population user data of the target population;
determining, by one or more processors, that the population criteria for the target population is above a first threshold population criteria;
receiving initiated population data for an initiated population; and
determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount.

2. The method of claim 1, wherein:
the target population is based on a user data associated with a geographical area.

3. The method of claim 2, wherein:
the target population is further based on user data demographics and Risk Adjustment Scores.

4. The method of claim 1, wherein:
the population criteria is output by a third machine learning model configured to receive the user data from plurality of databases and output the population criteria based on the user data.

5. The method of claim 4, wherein:
the population criteria is a ratio of health events per predetermined number of individuals for the target population.

6. The method of claim 1, wherein
the improvement probability is based on comparing the target population criteria with a comparison population criteria improvement.

7. The method of claim 6, wherein:
the comparison population is a simulated population.

8. The method of claim 7, wherein:
the simulated population includes historical population data from one or more previously initiated target populations.

9. The method of claim 1, wherein:
the second threshold amount is a criteria minimum improvement to the population criteria to reach the first threshold population criteria.

10. A system for outputting new target populations for system initiation using a first machine learning model and a second machine learning model; the system comprising;
at least one memory storing instructions; and
at least one processor executing the instructions to perform a process including:
receiving, by one or more processors, user data associated with users from an external server via a secure network connection;
storing, by the one or more processors, the user data on a cloud-based data storage system;
identifying, by one or more processors, a target population based on the user data to find groups of individuals not currently serviced by a specific provider, wherein the target population is output by the machine learning model that receives the user data, applies the user data to one or more of first model weights, first model biases, or first model layers, and outputs the target population;
identifying, by one or more processors, a population criteria for the target population;
applying a population criteria to the target population user data of the target population;
determining, by one or more processors, that the population criteria for the target population is above a first threshold population criteria;
receiving initiated population data for an initiated population; and
determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount.

11. The system of claim 10, wherein:
the target population is based on a user data associated with a geographical area.

12. The system of claim 11, wherein:
the target population is further based on user data demographics and Risk Adjustment Scores.

13. The system of claim 10, wherein:
the population criteria is output by a third machine learning model configured to receive the user data from plurality of databases and output the population criteria based on the user data.

14. The system of claim 13, wherein:
the population criteria is a ratio of health events per predetermined number of individuals for the target population.

15. The system of claim 10, wherein:
the improvement probability is based on comparing the target population criteria with a comparison population criteria improvement.

16. A method for outputting new target population for system initiation using a first machine learning model and a second machine learning model, the method comprising:
receiving, by one or more processors, user data associated with users from an external server via a secure network connection;
storing, by the one or more processors, the user data on a cloud-based data storage system;
identifying, by one or more processors, a population criteria for target populations;
identifying, by one or more processors, a target population based on the user data and criteria to find groups of individuals not currently serviced by a specific provider, wherein the target population is output by the machine learning model that receives the user data, applies the user data to one or more of first model weights, first model biases, or first model layers, and outputs the target population;
determining, by one or more processors, that the population criteria for the target population is above a first threshold population criteria; and
determining, by one or more processor, that the system initiation meets an improvement threshold probability to improve the population criteria by a second threshold amount, wherein the determining is based on the second machine learning model that receives, as inputs, the target population user data and initiated population data, applies the user data to one or more of second model weights, second model biases, or second model layers, and outputs an improvement probability that the system initiation would improve the population criteria for the target populations by the second threshold amount.

17. The method of claim 16, wherein:
the improvement probability is based on comparing the target population criteria with a comparison population criteria improvement.

18. The method of claim 16, wherein:
the population criteria is output by a third machine learning model configured to receive the user data from plurality of databases and output the population criteria based on the user data.

19. The method of claim 16, wherein:
the second threshold amount is a criteria minimum improvement to the population criteria to reach the first threshold population criteria.

20. The method of claim 19, wherein:
the population criteria is a ratio of health events per predetermined number of individuals for the target population.
